(19) United States
(12) Patent Application Publication
(10) Pub. No.: US 2007/0196442 A1
(43) Pub. Date: Aug. 23, 2007

(54) Method for improving the oral administration of alpha-lipoic acid

(76) Inventor: Marvin A. Heuer, Mississauga (CA)

Correspondence Address: KENYON & KENYON LLP, ONE BROADWAY, NEW YORK, NY, 10004, US

(21) Appl. No.: 11/704,586

(22) Filed: Feb. 9, 2007

Related U.S. Application Data
(60) Provisional application No. 60/772,296, filed on Feb. 10, 2006.

(51) Int. Cl. A61K 31/385; A61K 47/00

(52) U.S. Cl. 424/439; 514/440

(57) ABSTRACT

The invention provides a method for reducing esophageal irritation associated with alpha-lipoic acid upon oral administration through the use of the trometamol salt of alpha-lipoic acid. The present invention also provides for the increased solubility of alpha-lipoic acid through the use of the trometamol salt of alpha-lipoic acid. The trometamol salt of alpha-lipoic acid, as provided by the present invention can be used as a substitute for regular, non-salt forms alpha-lipoic acid in dietary supplement compositions.

### RELATED APPLICATIONS

This application claims the benefit of priority of Applicant's co-pending U.S. Provisional Patent Application Ser. No. 60/772,296, filed on Feb. 10, 2006, the disclosure of which is hereby fully incorporated by reference.

### FIELD OF THE INVENTION

The invention relates to a method for the improved oral administration of alpha-lipoic acid. The present invention comprises the oral administration of the trometamol salt of alpha-lipoic acid.

### SUMMARY OF THE INVENTION

The present invention relates to a method of reducing the esophageal irritation associated with oral administration of alpha-lipoic acid. Another aspect of the present invention is to improve the aqueous solubility of alpha-lipoic acid.

### BACKGROUND OF THE INVENTION

Thioctic acid, more commonly known as alpha-lipoic acid is both an antioxidant and a modulator of insulin. It has known protective effects against oxidative stress and injury in neuronal and non-neuronal tissue. The human body is capable of manufacturing alpha-lipoic acid in minute quantities. Moreover, alpha-lipoic acid can be extracted from yeast and liver.

The present invention comprises the oral administration of the trometamol salt of alpha-lipoic acid, which may also protect against oxidative stress through the use of antioxidants in formulations designed for weight loss. Lipid peroxidation produces malondialehyde as well as reactive oxygen species (Arivazhagan P, Juliet P, Panneerselvam C. Effect of dl-alpha lipoic acid on the status of lipid peroxidation and antioxidants in aged rats. Pharmacol Res. 2000 March; 41 (3):299-303). Further to these effects, alpha-lipoic acid has been linked to an increase in High-Density Lipoprotein levels, thereby helping to scavenge cholesterol and decrease the total amount of deposited cholesterol and lipids in addition to its antioxidants properties (Wollin S D, Wang Y, Kubow S, Jones P J. Effects of a medium chain triglyceride oil mixture and alpha lipoic acid diet on body composition, antioxidant status, and plasma lipid levels in the Golden Syrian hamster. J Nutr Biochem. 2004 July; 15(7):402-10).

In addition to acting as an antioxidant, alpha-lipoic acid also possesses a two-fold mechanism related to hunger and β-oxidation of fat. AMP-activated protein kinase acts as a fuel level sensor within the hypothalamus. Alpha-lipoic Acid is a cofactor of mitochondrial enzymes which cause profound weight loss via a decrease in AMP-activated protein kinase in the hypothalamus of rodents. This decrease in hypothalamic AMP-activated protein kinase leads to a decrease in hypothalamic mitochondrial β-oxidation, resulting in reduced food intake (Kim M S, Park J Y, Namkoong C, Jang P G, Ryu J W, Song H S, Yun J Y, Namgoong I S, Ha J, Park I S, Lee I K, Viollet B, Youn J H, Lee H K, Lee K U. Anti-obesity effects of alpha lipoic acid mediated by suppression of hypothalamic AMP-activated protein kinase. Nat. Med. 2004 July; 10(7):727-33). A secondary action of alpha-lipoic acid relating to this mechanism is an increase in alpha-lipoic acid resulting in an increase in Uncoupling Protein-1 (UCP-1) expression in rodent adipocytes and an increase AMP in skeletal muscle respectively (Lee W J, Koh E H, Won J C, Kim M S, Park J Y, Lee K U. Obesity: the role of hypothalamic AMP-activated protein kinase in body weight regulation. Int J Biochem Cell Biol. 2005 November; 37(11):2254-9). Although UCP-1 is present in rodents, it is believed that homologues exist in humans, which utilize the same mechanism. These effects are also mediated by alpha-lipoic acid-induced hypothalamic inhibition of AMP-activated protein kinase (Kim M S, Park J Y, Namkoong C, Jang P G, Ryu J W, Song H S, Yun J Y, Namgoong I S, Ha J, Park I S, Lee I K, Viollet B, Youn J H, Lee H K, Lee K U. Anti-obesity effects of alpha lipoic acid mediated by suppression of hypothalamic AMP-activated protein kinase. Nat. Med. 2004 July; 10(7):727-33). The end-result of these actions is an increase in fatty acid oxidation from adipose tissue and in skeletal muscle, which leads to an increase energy expenditure and a decrease in weight (Lee W J, Koh E H, Won J C, Kim M S, Park J Y, Lee K U. Obesity: the role of hypothalamic AMP-activated protein kinase in body weight regulation. Int J Biochem Cell Biol. 2005 November; 37(11):2254-9) in addition to a decrease in food intake.

In addition to alpha-lipoic acid's effect with respect to weight loss, it has also been found to enhance glucose uptake into skeletal muscle in animal models (Burke D G, Chilibeck P D, Parise G, Tarnopolsky M A, Candow D G. Effect of alpha lipoic acid combined with creatine monohydrate on human skeletal muscle creatine and phosphagen concentration. Int J Sport Nutr Exerc Metab. 2003 September; 13(3):294-302). Moreover, in studies in which alpha-lipoic acid was administered in treatment groups, an increase in glucose transporter proteins (GLUT4) was observed (Estrada D E, Ewart H S, Tsakiridis T, Volchuk A, Ramlal T, Tritschler H, Klip A. Stimulation of glucose uptake by the natural coenzyme alpha lipoic acid/thioctic acid: participation of elements of the insulin signaling pathway. Diabetes. 1996 December; 45(12):1798-804). These data show that alpha-lipoic acid can aid in blood glucose clearance. Furthermore, studies have shown the alpha-lipoic acid can significantly increase the body's utilization of blood glucose in type II diabetes. These studies also show that alpha-lipoic acid may increase the metabolic clearance rate of glucose by 50% in diabetics.

Alpha-lipoic acid, although shown to have beneficial effects when administered does have drawbacks related to consumption. A preferred route of administration for alpha-lipoic acid is oral; however it has long been known that severe irritation of the upper potion of the esophagus, namely the throat occurs via this administration route.

Owing to its pH of 10.4 in a 0.1M aqueous solution, it often used as an alkalizer in the preparation of injectable drugs to obtain approximately physiological pH.

The inventors have noted that trometamol can be reacted with alpha-lipoic acid to form a salt with a pH of approximately 6.5 to 7.5. As physiological pH is 7.4, this salt is suitable for ingestion.

A method of production of tris(hydroxymethyl) amino methane thioctate, the trometamol salt of alpha-lipoic acid, is disclosed in U.S. Pat. No. 3,562,273 incorporated herein in it's entirely by reference. The patent discloses that the trometamol salt of alpha-lipoic acid is very soluble in water and has a pH of 6.5 in a 0.1M aqueous solution.

Alpha-lipoic acid is commonly used in dietary supplements for the aforementioned purposes. However, owing to the severe throat irritation associated with the oral administration of alpha-lipoic, there have been a long felt need for methods to achieve the same results as alpha-lipoic administration, however without having the oral irritation.

### BRIEF DESCRIPTION OF THE FIGURE

FIG. 1 is bar graph showing subject's ranking of liking the samples standardized for 0.5% Alpha-lipoic acid, according to an embodiment of the present invention.

### DETAILED DESCRIPTION OF THE INVENTION

As used herein for the purposes of this disclosure, the terms “throat irritation” and “esophageal irritation” refer to a burning sensation in the throat of an individual upon oral administration of alpha-lipoic acid.

Alpha-lipoic acid is also know in the art as Heparlipon, Lipoic acid, Biletan, Thioctacid, Thioactidase, Thioctidase Tioctidasi, Lipothion, Thioctsan and 5-[(3R)-dithiolan-3-yl]pentanoic acid. Henceforth, for the purposes of this disclosure, the immediately preceding will collectively be termed “alpha-lipoic acid.”

For the purposes of this disclosure, the following abbreviations may be employed: ALA may refer to alpha-lipoic acid, TR-ALA may refer to the trometamol salt of alpha-lipoic acid and Na-ALA may refer to the sodium salt of alpha-lipoic acid.

Trometamol is a commonly used physiogical buffer. Trometamol is also known by the synonyms: Tham, Tris, TROMETHAMINE, Tris buffer, Trisamine, Trisaminol, Trispuffer, Tromethane, Pehanorm and 2-amino-2-(hydroxymethyl)propane-1,3-diol. Henceforth, for the purposes of this disclosure, the immediately preceding will collectively be termed “trometamol.”

Alpha-lipoic acid is acidic by nature. Owing to this property, when administered orally, it may cause a severe burning sensation in the throat and esophageal irritation of the subject. Moreover, the acidity of the solution can also lead to gastric upset in the subject. The trometamol salt of alpha-lipoic acid ameliorates these indications, therefore making the administration of a solution containing the trometamol salt of alpha-lipoic acid more acceptable to a subject, having a reduced irritation of the throat as compared to a solution containing alpha-lipoic acid.

The present invention comprises the trometamol salt of alpha-lipoic acid to decrease the esophageal irritation associated with oral administration of alpha-lipoic acid. Another aspect of the present invention is the improved aqueous solubility of alpha-lipoic acid when in the form of the trometamol salt.

A comparative taste-test was employed, following a double-blind, randomized protocol, in order to test the inventor's hypothesis that the use of the trometamol salt of alpha-lipoic acid may reduce the throat and esophageal irritation associated with the oral administration of alpha-lipoic acid.

Experimental Protocol

A “taste-test” was devised involving 18 subjects, following a double-blind, randomized design wherein neither the subjects nor the experimenters administering the test where privy to the sample given to the subject. As such, three different forms of alpha-lipoic acid were used and handled according to a color code: normal alpha-lipoic acid (ALA, blue), the trometamol salt of alpha-lipoic acid (TR-ALA, yellow) and the sodium salt of alpha-lipoic acid (Na-ALA, red). Na-ALA is known to be more soluble and palatable than ALA and was included for purposes of comparison.

The test solutions were standardized to an ALA content of (0.5%) and were given to test subjects in a double-blind, random procedure, one at-a-time. Between samples, subject was given soda cracker and water to cleanse their palate and eliminate and throat irritation that may have resulted from the administration of a prior sample. A 1 min period between sample administrations was allowed to lapse between the various samples being given to the subjects. Subjects were asked to consecutively rate each of the three unknown samples based on two subjective parameters.

First, subjects were asked to rate the degree of “Throat Irritation” (burning) according to a 10-point scale with “1” designated as “No Burning” and “10” designated as “Severe Burning”.

The second subjective rating was “Overall Liking”. This was assessed with one of nine designations: “Dislike Extremely”, “Dislike Very Much”, “Dislike Moderately”, “Dislike Slightly”, “Neither Like or Dislike”, “Like Slightly”, “Like Moderately”, “Like Very Much”, or “Like Extremely”.

Preparation of the Samples

As noted above, each of the samples, TR-ALA, Na-ALA and ALA were standardized to contain 0.5% w/v ALA. The standardization was based on the percent ALA in the given solutes. For example, the ALA solute contained 99% pure ALA, and the TR-ALA solute was 50% pure ALA. The samples were standardized to 0.5% based on these values.

Owing to ALA's poor aqueous solubility, all solutions were maintained in a beaker on a magnetic stirrer for the duration of the experiment. The samples administered to subjects were extracted from the stock beakers with 1 ml disposable pipettes and given to the subjects. The subjects were then asked to squeeze the bulb of the pipette with the tip in their mouth, thus dispensing the solution into the mouth towards the throat.

Statistical Analysis

The Student's T-Test was use to analyze the subject's response to the samples and compared against one another.

Results:

The tabulated results for the taste test are presented in Table 1.

TABLE 1
ALA taste test data for subjective measures.

| | Sample 1 (blue) | | Sample 2 (yellow) | | Sample 3 (red) | |
|---|---|---|---|---|---|---|
| | ALA | | ALA-TR | | Na-ALA | |
| Subject | BURNING | OVERALL | BURNING | OVERALL | BURNING | OVERALL |
| 1 | 7 | Dislike | 1 | Like Slightly | 3 | Dislike Mod. |
| | | Extrem. | | | | |
| 2 | 6 | Dislike | 1 | Like Slightly | 2 | Neither |
| | | Slightly | | | | |
| 3 | 9 | Dislike Very | 4 | Dislike Mod. | 3 | Dislike Mod. |
| | | Much | | | | |
| 4 | | | 8 | Dislike Very | 10 | Dislike |
| | | | | Much | | Extrem. |
| 5 | 6 | Dislike Mod. | 4 | Dislike | 2 | Like Slightly |
| | | | | Slightly | | |
| 6 | 5 | Neither | 2 | Dislike Mod. | 1 | Neither |
| 7 | 9 | Dislike Very | 7 | Dislike Mod. | 5 | Neither |
| | | Much | | | | |
| 8 | 6 | Dislike | 8 | Dislike Mod. | 5 | Neither |
| | | Slightly | | | | |
| 9 | 3 | Dislike Mod. | 2 | Neither | 4 | Dislike Mod. |
| 10 | 3 | Dislike Very | 1 | Neither | 1 | Like Slightly |
| | | Much | | | | |
| 11 | 8 | Dislike Mod. | 7 | Dislike | 3 | Neither |
| | | | | Slightly | | |
| 12 | 9 | Dislike | 7 | Dislike Very | 3 | Dislike Mod. |
| | | Extrem. | | Much | | |
| 13 | 6 | Dislike Mod. | 6 | Dislike Mod. | 5 | Dislike Mod. |
| 14 | 3 | Dislike Mod. | 2 | Dislike Mod. | 2 | Dislike Mod. |
| 15 | 6 | Dislike Very | 2 | Dislike | 4 | Dislike Mod. |
| | | Much | | Slightly | | |
| 16 | 6 | Neither | 1 | Dislike | 1 | Dislike Very |
| | | | | Slightly | | Much |
| 17 | 8 | Dislike | 5 | Dislike Mod. | 7 | Dislike Very |
| | | Extrem. | | | | Much |
| 18 | 5 | Dislike | 3 | Neither | 2 | Neither |
| | | Slightly | | | | |

Tables 2-4 present the means and statistical analysis (t-test) of various relevant comparisons.

TABLE 2
ALA VS. TR-ALA

| | ALA | TR-ALA |
|---|---|---|
| Mean | 6.176471 | 3.944444 |
| Variance | 4.029412 | 6.879085 |
| Observations | 17 | 18 |
| P value | 0.008168 | |

TABLE 3
ALA VS. Na-ALA

| | ALA | Na-ALA |
|---|---|---|
| Mean | 6.176471 | 3.5 |
| Variance | 4.029412 | 5.323529 |
| Observations | 17 | 18 |
| P value | 0.000893 | |

TABLE 4
TR-ALA VS. Na-ALA

| | TR-ALA | Na-ALA |
|---|---|---|
| Mean | 3.944444 | 3.5 |
| Variance | 6.879085 | 5.323529 |
| Observations | 18 | 18 |
| P value | 0.592858 | |

The statistical analysis with respect to the subject's rankings of throat irritation or burning show that there is a difference between the ALA and both of the TR-ALA and Na-ALA samples. The means for the TR-ALA and Na-ALA are 3.9 and 3.5 respectively, wherein they are less than that of 6.1 for ALA on the ranking scale showing less throat irritation is noted with the TR-ALA and Na-ALA solutions. Surprisingly a statistical analysis between the throat irritation noted for the TR-ALA versus the Na-ALA sample shows no difference.

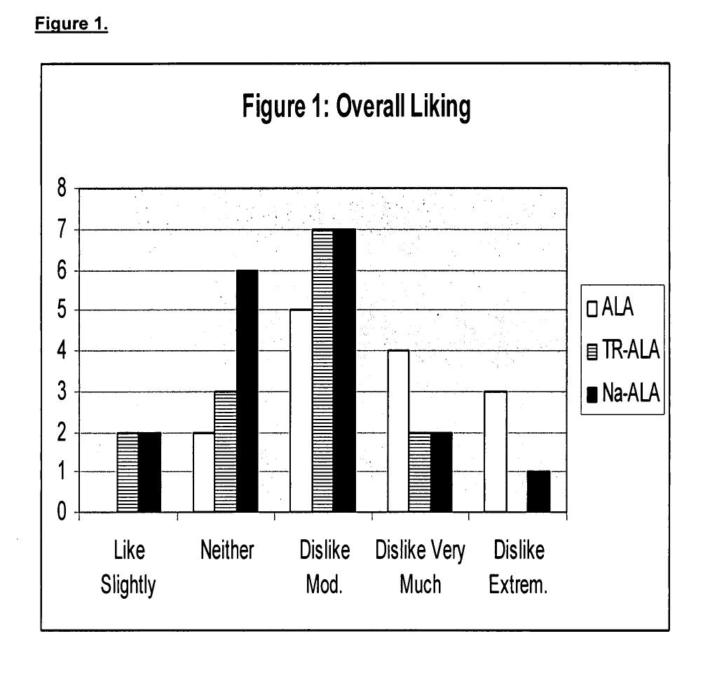

The responses for rating the samples in terms of “Overall Liking” were tabulated and are presented in FIG. 1. From FIG. 1, it can be seen that subjects preferred the Na-ALA and the TR-ALA as compared to the ALA.

Discussion

As can be seen from the data in Tables 2 and 3, there was a significant difference between the subjective ranking of ALA compared to both TR-ALA and Na-ALA in terms of “Throat Irritation” with ALA being more irritating to the throat. As can also be seen from Table 4, there was no significant difference between TR-ALA and Na-ALA in terms of “Throat Irritation”. This, surprisingly to the inventor indicates that TR-ALA reduced the throat irritation associated with alpha-lipoic acid in similar fashion to NA-ALA. The inventors have thus shown that TR-ALA can be effectively used to reduce the throat irritation upon oral administration of alpha-lipoic acid.

From the trends in the graph in FIG. 1, “Overall Liking” was most favorable, in terms of ‘least disliked’ for TR-ALA, with no subjects assigning “Dislike Extremely” (the lowest rank available). In comparison, one subject ranked Na-ALA and three subjects ranked ALA with “Dislike Extremely”. In terms of ‘most liked’, two subjects each assigned TR-ALA and Na-ALA with “Like Slightly”, which was the ‘most liked’ category selected from those available. No subjects assigned ALA “Like Slightly”.

Since the order of sampling was random, each subject may have a different reference point for what constitutes ‘burning’. Therefore, it may be useful to examine the trends among the subjects, which should be similar across subjects for all samples. One of the eighteen subjects failed to rank the ALA sample, and will therefore not be considered. Of the remaining seventeen subjects, fourteen ranked the ALA sample as the most irritating of the three samples. Conversely, five of the seventeen ranked TR-ALA as the least irritating of the three samples, nine of the seventeen ranked Na-ALA as the least irritating, while two ranked both TR-ALA and Na-ALA of equal irritation (in both cases lower than ALA). However the statistical analysis indicated that there is no difference in the throat irritation between the TR-ALA and the Na-ALA, however, both are significantly less irritating to the throat of the subjects as compared to ALA.

Another variable of note is that due to the subjective nature of the test and ranking, individual tolerance of what constitutes “irritation” may affect the range of the numerical values. Likewise, the contribution of irritation, or lack thereof, may contribute to overall liking to different degrees among individuals. Overall liking is expected to be influenced by factors other than irritation such as taste, flavor and texture.

Based upon the taste test results depicted in Tables 1-4 and FIG. 1, and the preceding discussion, the trometamol salt of alpha-lipoic acid surprisingly offers a reduction in the esophageal or throat irritation that normally associated with regular alpha-lipoic acid when administered orally. The improvement offered is comparable to that offered by the sodium salt of alpha-lipoic acid.

The inventors also noted that the trometamol salt of alpha-lipoic dissolved better in the aqueous solution that alpha-lipoic acid at the dosage tested. The inventors understand that this would be case for either higher or lower dosages of the trometamol salt of alpha-lipoic acid compared to similar doses of alpha-lipoic acid. Surprisingly, the inventors have observed that at comparable concentrations of alpha-lipoic acid, the trometamol salt of alpha-lipoic has improved solubility in aqueous mediums as comparable to non-salt forms of alpha-lipoic acid.

The present invention is useful in the following example embodiments of orally administered dietary supplements which comprise the use of the of alpha-lipoic acid. Alpha-lipoic acid as disclosed in U.S. Pat. Nos. 6,136,339, 6,420,425, and Canadian Patent No. 2,246,014, incorporated herein in their entirety by reference can be substituted with the trometamol salt of alpha-lipoic acid, to reduce the throat irritation associated with oral administration of alpha-lipoic acid. With respect to these compositions, poorly-soluble, alpha-lipoic acid, which causes a burning sensation and throat or esophageal irritation upon ingestion, may be substituted with the trometamol salt of alpha-lipoic acid to increase solubility of alpha-lipoic acid in the resultant solutions when the compositions are mixed with water. Furthermore, the uses of trometamol alpha-lipoic acid in these compositions will reduce the throat or esophageal irritation or burning sensation associated with non-salt forms of alpha-lipoic acid upon oral administration. Example embodiments of incorporating the present invention are set forth in greater detail in Examples 1 to 5.

Although the following examples illustrate the practice of the present invention in 5 potential embodiments, the examples should not be construed as limiting the scope of the invention. Other embodiments will be apparent to one of skill in the art from consideration of the specification and the following examples.

### EXAMPLE 1

A serving of the dietary supplement comprises the following ingredients in powdered beverage mix form. The dietary supplement may, for example, be mixed in 12 oz. of cold water. After finishing the preparation, drink an additional 8 oz. of water to help enhance absorption. The dietary supplement comprises for example: Dextrose 99 DE (83.69 g), Creatine monohydrate (9.85 g), Taurine (2.3 g), Dipotassium phosphate (0.33 g), Disodium phosphate 98% (0.33 g), Magnesium phosphate 98% (0.33 mg), Vitamin C (0.25 g), Alpha-lipoic acid Trometamol (0.22 g), Betaine HCl (0.1 g), Glutamine AKG 2:1 (0.1 g), Creatine anhydrous (50 μg), Creatine AKG 2:1 (50 μg), Creatine malate (50 μg), Creatine ethyl ester HCl (5 μg) and Chromium polynicotinate (0.3 μg). It is recommended for best results that a serving of the dietary supplement, as described, be consumed one to four times daily, for eight weeks.

### EXAMPLE 2

A serving of the dietary supplement comprises the following ingredients in powdered beverage mix form. The dietary supplement may, for example, be mixed with 12 to 14 oz. of cold water or skim milk and mixed in a blender or shaker for 30 seconds. The dietary supplement comprises for example: Whey protein concentrate 80% instant (56.5 g), Maltodextrin 10DE (10 g), Creatine monohydrate (3 g), Oat fiber (1.4 g), Postassium citrate (1 g), Potassium chloride (0.64 g), Calcium caseinate (0.33 g), Whey protein isolate 97% ion-exchange (0.26 g), Milk protein concentrate (0.25 g), Magnesium oxide (0.12 g), Vitamin mineral premix (107 μg), Egg albumin (100 μg), Choline bitartrate (100 μg), Konjac flour (100 μg), Brown rice powder (100 μg), Inulin IQ (10 μg), Micellar casein (10 μg), encapsulated Flaxseed oil powder (10 μg), Borage oil powder (10 μg), L-lysine (1 μg), L-valine (1 μg), L-isoleucine (1 μg), L-methionine (1 μg), L-arginine (1 μg), L-arginine alpha-ketolglutarate (1 μg), L-glutamine (1 μg), Glutamine alpha ketoglutarate 2:1 (1 μg), L-leucine (1 μg), L-leucine ethyl ester (1 μg), L-phenylalanine (1 μg), L-threonine (1 μg), L-histidine (1 μg), Taurine (1 μg), L-leucine ethyl ester (1 μg), Citrulline (1 μg), Alpha-lipoic acid Trometamol (1 μg), Fish oil (1 μg) and Lactoferrin (360 ng). A serving of the dietary supplement, as described, may be consumed one to four times daily.

### EXAMPLE 3

A serving of the dietary supplement comprises the following ingredients in powdered beverage mix form. The dietary supplement may, for example, be mixed with 6 oz. of cold water and consumed immediately. The dietary supplement comprises for example: Dextrose 99 DE (45.5 g), Creatine monohydrate (5.9 g), Taurine (1.72 g), Alpha-lipoic acid as the Trometamol salt (0.28 g), L-Glutamine (0.1 g), Dipotassium phosphate (0.1 g), Disodium phosphate (0.1 g), Magnesium phosphate (0.1 g), Creatine pyruvate (0.05 g), Creatine citrate (0.05 G), L-Leucine (1.6 μg), L-Valine (1.6 μg), L-Isoleucine (1.6 μg) and L-Glycine (1.6 μg). A serving of the dietary supplement, as described, may be consumed one to four times daily.

### EXAMPLE 4

A serving of the dietary supplement comprises the following ingredients in powdered beverage mix form. The dietary supplement may, for example, be mixed with 6 oz. of cold water and consumed immediately. The dietary supplement comprises for example: WPC-80, instant (20.33 g), WPI-97 (2.01 g), Creatine monohydrate, fine grind (2 g), Inositol (0.2 g), WPC-80, part hydrolyzed (0.16 g), Taurine (0.1 g), Konjac flour (0.1 g), Dipotassium phosphate (0.1 g), Magnesium oxide (0.05 g), L-Citrulline (0.05 g), N-acetyl cysteine (0.03 g), American ginseng extract 5% (0.02 g), Alpha-lipoic acid Trometamol (0.02 g), Glutamine AKG (2 μg), D-Pinitol (2 μg), L-Leucine (1.6 μg), L-Methionine (1.6 μg), L-Phenylalanine (1.6 μg), L-Lysine (1.6 μg), L-Threonine (1.6 μg), L-Valine (1.6 μg), L-Isoleucine (1.6 μg), L-Histidine (1.6 μg), Calcium AKG (1.6 μg), Lactoferrin (0.2 μg) and Folic acid (0.17 μg). A serving of the dietary supplement, as described, may be consumed two to six times daily.

### EXAMPLE 5

A serving of the dietary supplement comprises the following ingredients in powdered beverage mix form. The dietary supplement may, for example, be mixed with 16 to 18 fluid ounces of cold water or skim milk, mixed in a blender or shaker for 30 seconds and consumed between meals and following exercise one to four times daily. The dietary supplement comprises for example: Whey Protein Concentrate (10.6 g), Vitamin A (2500 IU), Vitamin C (30 mg), Vitamin E (15 IU), Riboflavin (0.85 mg), Thiamin (0.75 mg), Folic Acid (0.2 mg), Vitamin B12 (3 g), Biotin (0.15 mg), Pantothenic Acid (5 mg), Creatine Monohydrate (2 g), Glutamine Peptides (0.028 g), L-Glutamine (0.028 g), L-Leucine (1.4 μg), L-Valine (1.4 μg), L-Isoleucine (1.4 μg), N-acetyl-cysteine (0.023 g), Alpha-lipoic acid as the Trometamol salt (0.015 g) and D-Pinitol (1.8 μg).

What is claimed:

1. A method for reducing esophageal irritation associated with the oral administration of alpha-lipoic acid, said method comprising the oral administration of a trometamol salt of alpha-lipoic acid to a mammal.

2. The method of claim 1 wherein said trometamol salt of alpha-lipoic acid comprises a dietary supplement.

3. The method of claim 2 wherein said trometamol salt of alpha-lipoic acid functions as an antioxidant.

4. The method of claim 2 wherein said trometamol salt of alpha-lipoic acid functions to increase glucose transporter proteins.

5. The method of claim 2 wherein said trometamol salt of alpha-lipoic acid functions to increase the metabolic clearance rate of glucose.

6. The method of claim 2 wherein said trometamol salt of alpha-lipoic acid reduces muscle recovery time in a mammal.

7. The method of claim 2 wherein said trometamol salt of alpha-lipoic acid improves recovery time in a mammal.

8. The method of claim 2 wherein said trometamol salt of alpha-lipoic acid enhances weight loss in a mammal.

9. The method of claim 2 wherein said trometamol salt of alpha-lipoic acid enhances fat loss in a mammal.

10. A method for improving the solubility of alpha-lipoic acid included in aqueous dietary supplements comprising the use of the trometamol salt of alpha-lipoic acid.

11. A dietary supplement for ingestion orally by a mammal, the dietary supplement comprising:
a trometamol salt of alpha-lipoic acid.
